(19) United States
(12) Patent Application Publication
(10) Pub. No.: US 2004/0229230 A1
(43) Pub. Date: Nov. 18, 2004

(54) Method for gene isolation by Cre-trap cloning

(76) Inventor: Barry P. Sleckman, St. Louis, MO (US)

Correspondence Address: Scott J. Meyer - MC5S, Pharmacia Corp., Corporate Patent Dept., P.O. Box 1027, St. Louis, MO, 63006, US

(21) Appl. No.: 10/643,508

(22) Filed: Aug. 19, 2003

Related U.S. Application Data
(60) Provisional application No. 60/406,152, filed on Aug. 27, 2002.

(51) Int. Cl. C12Q 1/68; C12N 15/85

(52) U.S. Cl. 435/6; 435/455; 435/320.1

(57) ABSTRACT

The disclosure describes a method for isolating genes encoding proteins that regulate the expression of target genes of interest, said method designated herein for convenience by the term: “Cre-Trap cloning.” In brief, the Cre-trap cloning method of the invention involves modifying a target gene of interest such that it encodes the Cre recombinase and Herpes simplex virus (HSV) thymidine kinase (TK) proteins. This step is followed by mutagenesis and selection for cells which have lost target gene expression by virtue of their resistance to ganciclovir (a nucleoside analog structurally related to acyclovir), which kills cells expressing HSV TK. Cell lines that have lost target gene expression due to mutations in genes encoding trans-acting factors are then transiently transfected with a cDNA library from the parent cell line in a novel expression vector (pCT.1, shown in FIG. 1). Expression of a cDNA that complements the genetic mutation results in expression of the target gene and production of the Cre recombinase, which modifies pCT.1 in such a way that it is readily isolated from pCT.1 plasmids with cDNAs that do not activate target gene expression.

This application claims the benefit of application Ser. No. 60/406,152, filed Aug. 27, 2002.

### FIELD OF THE INVENTION

The present invention relates to the field of gene isolation by cloning.

### BACKGROUND OF THE INVENTION

The development of novel methods for isolating genes encoding proteins that regulate the expression of target genes of interest is of importance for identifying and obtaining therapeutic targets for novel drugs.

A conventional method for such purpose is one-hybrid-cloning. One-hybrid-cloning has been described by Wang and Reed, Nature, 1993, 364; 121-6; Luo et al., Biotechniques, 1996, 20, 564-8; and Hasegawa et al., J. Biol. Chem., 1997, 272, 4915-4923 (refs. 1-3, below).

In brief, the one-hybrid-cloning method is a yeast cloning system in which a cis-acting element for the gene of interest is placed upstream of a reporter gene. A cDNA library fused to a sequence encoding a transcriptional trans-activating protein is introduced into the yeast, and then cDNAs encoding protein that can bind the cis-acting element from the gene of interest are isolated based on transcriptional activity of the reporter gene.

A drawback of the conventional one-hybrid cloning method is that it is limited to the isolation of genes encoding proteins that can directly and independently bind to a defined cis-acting element. This is a drawback as many proteins that regulate gene expression through direct interaction with cis-acting elements do so only as part of a multi-protein complex and will not bind in the absence of the other distinct proteins in the complex. In addition, there are many proteins that regulate gene expression indirectly in ways that do not require direct binding to cis-acting elements. The genes encoding these proteins would not be isolated using the one-hybrid cloning method. Accordingly, it is desired to develop an improved method for isolating genes that encode proteins that regulate expression of genes of interest.

### BRIEF DESCRIPTIONS OF THE INVENTION

In accordance with the present invention a novel method is provided for isolating genes encoding proteins that regulate the expression of target genes of interest. For convenience, this novel method is designated by the term: “Cre-trap cloning.” In brief, the Cre-trap cloning method of the invention involves modifying a target gene of interest such that it encodes the Cre recombinase and Herpes simplex virus (HSV) thymidine kinase (TK) proteins. This step is followed by mutagenesis and selection for cells which have lost target gene expression by virtue of their resistance to ganciclovir (a nucleoside analog structurally related to acyclovir), which kills cells expressing HSV TK. Cell lines that have lost target gene expression due to mutations in genes encoding trans-acting factors are then transiently transfected with a cDNA library from the parent cell line in a novel expression vector (pCT.1, shown in FIG. 1). Expression of a cDNA that complements the genetic mutation results in expression of the target gene and production of the Cre recombinase, which modifies pCT.1 in such a way that it is readily isolated from pCT.1 plasmids with cDNAs that do not activate target gene expression.

The method of the invention, which does not require that the cis-acting elements be defined, nevertheless can yield clones for proteins that directly bind cis-acting elements. Unlike one-hybrid-cloning, the method of the present invention can also be used to identify genes important for gene expression due to effects that may not be exerted directly at the target gene. Such proteins can be part of the genetic program of a cell and as such can serve as excellent drug targets aimed at altering cellular genetic programs involved in pathologic conditions.

The method of the invention thus utilizes in part the Cre recombinase technology described by Sauer and Henderson, Nucleic Acids Res., 1989, 17: 147-61; Sauer, Mol. Cell Biol., 1987, 7: 2087-2096; and Sauer and Henderson, Proc. Natl. Acad. Sci. U.S.A., 1988, 85: 5166-70 (refs. 4-6). See also Sauer, U.S. Pat. No. 4,959,317.

A review article on the conventional Cre recombinase system also has been published by Andras Nagy, “Cre Recombinase: The Universal Reagent for Genome Tailoring”, Genesis, 2000, 26, 99-109 (ref. 17). The Cre recombinase of the P1 bacteriophage is a 38 kilodalton protein that catalyzes the recombination between two of its recognition sites, called loxP. The loxP site is a 34-base pair sequence composed of two 13-base-pair inverted repeats separated by an asymmetric 8-base-pair core sequence.

### DETAILED DESCRIPTION OF THE INVENTION

While the specification concludes with claims particularly pointing out and distinctly claiming the subject matter regarded as forming the invention, it is believed that the invention will be better understood from the following preferred embodiments taken in conjunction with the accompanying drawings.

### BRIEF DESCRIPTION OF THE DRAWINGS

FIG. 1: Schematic of the pCT.1 vector in the configurations that impart ampicillin resistance (pCT.1^{AMP}) and chloramphenicol resistance (pCT.1^{CAT}). Shown is the chloramphenicol resistance gene (CAT) and ampicillin resistance gene (βLAC) with arrows indicating their transcriptional orientation. Also shown are the loxP sites (triangles) which are positioned as inverted repeats, the gene encoding polyoma large T antigen (Poly LT) and the polyoma origin of replication (Por). Eukaryotic (filled circle) and prokaryotic (open circle) promoters are shown, as is the position of the multiple cloning site (MCS) for cloning cDNAs.

FIG. 2: Schematic of retroviral vector showing the long terminal repeats (LTRs), upstream activating sequence (UAS) binding site for Gal4, cDNA for the GFPCre fusion protein, independent ribosomal entry site (IRES) and cDNA of the herpes simplex virus (HSV) thymidine kinase (TK) gene.

FIG. 3: Flow cytometric analyses of BW cell lines expressing the GFPCre:IRES:HSVTK retroviral cassette (FIG. 2). Shown is the parent BW5147 murine thymoma cell line (BW) and four of the GFPCre expressing clones (BW-1, 5, 41 and 50).

FIG. 4: Schematic of clone capture and enrichment using the BWm mutant cell lines. The BWm mutant cell lines have two copies of the retroviral GFPCre:TRES:HSVTK cassette (FIG. 2) with mutations in both LTRs or two copies of a trans-acting factor required for expression from the LTR. As a result these cells do not express the GFPCre:TRES:HSVTK cassette. Transient expression of Gal4 from pCT.1^{AMP}-Gal4 results in expression of the GFPCre:TRES:HSVTK cassette and production of the GFPCre fusion protein which converts pCT.1^{AMP}-Gal4 to pCT.1^{CAT}-Gal4. Enrichment is carried out by serial transfections of recovered plasmids alternating bacterial selection with ampicillin and chloramphenicol for pCT.1^{AMP}/pCT.1^{AMP}-Gal4 and pCT.1^{CAT}/pCT.1^{CAT}-Gal4 plasmids, respectively.

FIG. 5: Schematic of the gene targeting step for a desired target gene. The targeting construct (pTarget/GFPCre:IRES:HSVTK) and a schematic of targeted locus (Target/GFPCre:IRES:HSVTK) are shown. The GFP:IRES:HSVTK cassette is described in the description of FIG. 2. The neomycin resistance gene (Neo) flanked by FRT sites (open circles) is shown. The UAS (filled oval) and the target gene promoter (arrow) are indicated. The non-coding (shaded) and coding (filled) portions of the exons are shown.

FIG. 6: Analysis of ganciclovir resistant mutants. Ganciclovir resistant mutants that have mutations in both copies of the HSVTK gene or target gene cis-acting elements on both alleles are eliminated based on expression of GFPCre or failure to rescue GFPCre:IRES:HSVTK expression upon fusion to the parent cell lines. Expression of the GFPCre:IRES:HSVTK cassette in cell lines with mutations in both copies of a gene encoding a target gene trans-acting factor is rescued upon fusion to the parent cell line.

In order to further illustrate the invention in greater detail, the following specific Examples are provided. Although specific examples are thus illustrated herein, it will be appreciated that the invention is not limited to these specific, illustrative examples or the details therein. These specific Examples are initially summarized for convenience as follows:

### EXAMPLE 1

### Generation of the Cre Trap Vector pCT.1

1.1 Construction of the pCT.1 Vector

1.2 Cre Modification of pCT.1

1.3 Episomal Replication of pCT.1

### EXAMPLE 2

### Test of the Cre Trap Cloning Method

2.1 GFPCre:IRES:HSVTK Cassette Function

2.2 Evaluation of Mutagenesis Step

2.3 Critical Parameters for Cre Trap Clone Capture

2.4 Critical Parameters for Cre Trap Clone Enrichment

### EXAMPLE 3

### Application of Cre Trap Cloning to a Target Gene

3.1 Generation of Cell Lines with GFPCre:IRES:HSVTK Targeted Loci

3.2 Isolation of Genes Encoding Proteins that Regulate Target Gene Expression

For convenience and reference, various scientific publications on conventional materials, laboratory procedures, and other state-of-the-art techniques and procedures commonly used and well-known to the person skilled in the art and employed in the following Examples are indicated by reference numerals in parentheses and cited at the end of the specification under the heading “References.”

For example, the gene for thymidine kinase (tk) is one of the most well known selective markers for transfection experiments. The transfer of purified herpes simplex virus thymidine kinase gene (HSV tk) to cultured mouse cells is conventional laboratory procedure as evident from appended reference 18.

Likewise BW5157 is a state-of-the-art murine lymphoma cell line, and AKR mice with BW5157 lymphatic leukemia are conventional as seen from reference 14. So also, polyoma virus (a small DNA tumor virus, 5292 bp) and the large T antigen are common materials, and plasmids containing the polyoma origin of replication and large T antigen are well known as described in reference 11.

Another small DNA tumor virus, SV40 (5243 bp) and the SV 40 large T antigen, as described in appended reference 12, can be analogously used in human cell lines. Various of the materials used herein are also commercially available. For example, the pBluescript SK vector pBSSK, is commercially available from Stratagene, La Jolla, Calif.

### EXAMPLE 1

### Generation of the Cre Trap Vector pCT.1

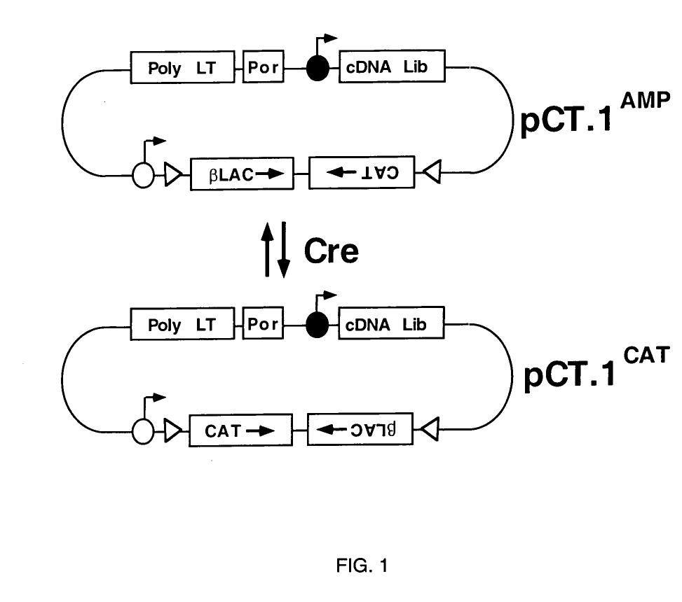

1.1 Construction of the pCT.1 vector: A novel eukaryotic expression vector, pCT.1 (FIG. 1) was developed. A critical feature of this vector is its ability to be modified by the Cre recombinase in a manner that will permit isolation from non-modified forms. The key components of pCT.1 are as follows:

a) pCT.1 has a eukaryotic promoter (FIG. 1, filled circle) for expression of cloned cDNAs in mammalian cell lines. This promoter can be changed to optimize expression in different cell lines.

b) pCT.1 has a cassette with convergently transcribed β-lactamase (Amp^{r}) and chloramphenicol acetyl transferase (CAT) genes (FIG. 1). The Amp^{r} and CAT genes impart bacterial resistance to ampicillin and chloramphenicol, respectively (refs. 7, 8). The β-lactamase promoter upstream of this cassette (FIG. 1, open circle) promotes sense transcripts of the Amp^{r} gene and anti-sense transcripts of the CAT gene (ref. 9). Accordingly, bacteria containing pCT.1 (also called pCT.1^{AMP}) are resistant to ampicillin and sensitive to chloramphenicol. There are two loxP sites: one between the α-lactamase promoter and the Amp^{r} gene and another 3′ of the CAT gene (FIG. 1, triangles). These loxP sites are positioned as inverted repeats, so the Cre recombinase catalyzes inversion of the Amp^{r}/CAT gene cassette (FIG. 1) (ref. 10). After Cre mediated inversion, the resulting plasmid, pCT.1^{CAT}, makes sense transcripts of the CAT gene and anti-sense transcripts of the Amp^{r} gene. As a result, pCT.1^{CAT} imparts bacterial resistance to chloramphenicol and not ampicillin (FIG. 1). As Cre mediated inversion regenerates functional loxP sites, pCT.1^{CAT} is readily converted back to pCT.1, which is important for the enrichment step (see 2.3).

c) pCT.1 contains the gene encoding Polyoma large T antigen and the polyoma origin of replication allowing for episomal replication in rodent cell lines (ref. 11). Versions of pCT.1 with the SV40 large T antigen gene and the SV40 origin can be used in human cell lines (ref. 12). In addition there is a bacterial origin of replication to allow for replication in bacteria (ref. 13).

The plasmid vector, pMG20Neo, (ref. 11), is a suitable plasmid as a source of the polyoma origin of replication and polyoma large T antigen gene in construction of pCT.1^{AMP}

1.2 Cre modification of pCT.1: A bacterial cell line expressing the Cre recombinase was transformed with pCT.1 (pCT.1^{AMP}) and the recovered plasmids were in the pCT.1^{AMP} and pCT.1^{CAT} configurations at an equimolar ratio. Furthermore, transformation of the recovered pCT.1^{AMP} or pCT.1^{CAT} also gave equimolar ratios of pCT.1^{AMP} and pCT.1^{CAT}. These findings demonstrate that the Amp^{r}/CAT gene cassette in pCT.1 is efficiently inverted by the Cre recombinase and that this reaction regenerates functional loxP sites that act as substrates for subsequent inversions.

1.4 Episomal replication of pCT.1: Episomal replication of pCT.1 in rodent cell lines occurs through the effect of Polyoma large T expression on the polyoma origin of replication. Episomal replication of pCT.1 results in higher expression levels of cloned cDNAs, more efficient plasmid recovery and increased probability of clone “capture” (see step 2.3). Equimolar ratios of pCT.1 and the non-replicating pBSSK plasmid were cotransfected into the murine thymoma, BW5147 (ref. 14). The plasmids recovered at 48 hours by Hirt extraction had an increased molar ratio of pCT.1 that was predominantly resistant to Dpn I digestion, thus demonstrating that pCT.1 had undergone de novo episomal replication in the BW5147 cell line (ref. 8).

### EXAMPLE 2

### Test of the Cre Trap Cloning Method

The Cre Trap Cloning method relies on the ability to genetically manipulate cells to express a bicistronic mRNA encoding the green fluorescent protein-Cre recombinase (GFPCre) fusion protein and the HSV thymidine kinase (TK) gene from the target gene of interest (refs. 15,16). A successful cloning method must be sensitive enough to isolate or “capture” a clone of interest during the initial cloning step and must be capable of enriching for these clones in subsequent steps. In this Example, the key components of Cre Trap Cloning are tested and the parameters for capture and enrichment determined.

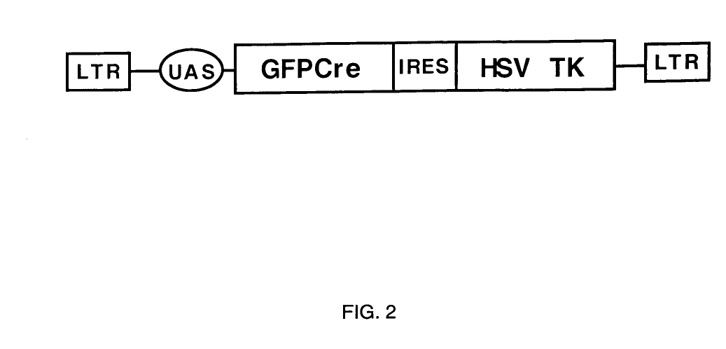

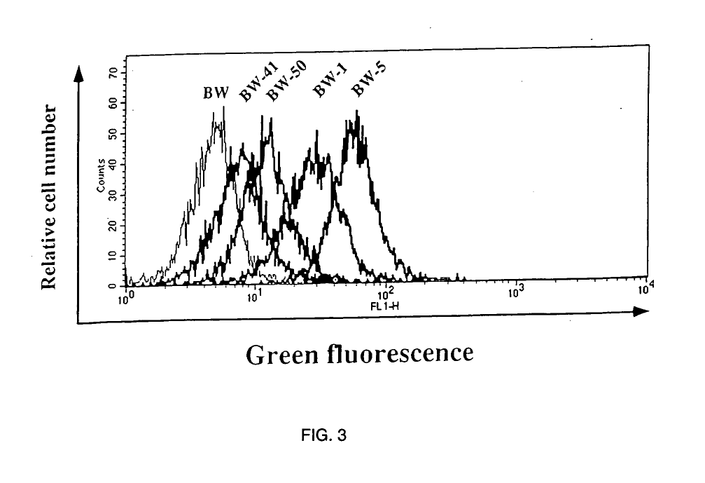

2.1 GFPCre:IRES:HSVTK cassette function: A retroviral vector was generated with the cDNA encoding the GFPCre fusion protein followed by an independent ribosomal entry site (IRES) and the cDNA encoding HSVTK (FIG. 2). In addition, the upstream activating sequence (UAS) which binds the Gal4 transcriptional trans-activator was cloned upstream of the GFPCre cDNA (FIG. 2). The BW5147 thymoma was infected with this retrovirus, and subclones (BW-1, 5, 41 and 50) expressing different levels of the GFPCre fusion protein were identified by flow cytometry (FIG. 3).

BW subclones with varying levels of GFPCre expression were tested for functional Cre recombinase and HSVTK activity. The pCT.1 (pCT.1^{AMP}) vector was transiently transfected into each cell line followed by recovery and analysis of plasmid configuration (pCT.1^{AMP} vs. pCT.1^{CAT}). Maximal conversion would result in equimolar ratios of pCT.1^{AMP} and pCT.1^{CAT} due to the reversible nature of the reaction. These analyses revealed that each of the GFPCre expressing BW cell lines was capable of maximal pCT.1 conversion, thus demonstrating that the GFPCre fusion protein has efficient Cre recombinase activity even at low cellular levels (BW-41, FIG. 3). The kinetics of conversion in cell lines expressing different levels of GFPCre was evaluated. To assess HSVTK activity, all of the cell lines were cultured in 5 μM ganciclovir. The parent BW cell line exhibited normal growth in media with 5 μM ganciclovir whereas the GFPCre:IRES:HSVTK expressing clones all died. No spontaneous revertants were isolated, which, given the culture conditions, puts the spontaneous reversion rate at <10⁻⁸. Together these findings demonstrate that the bicistronic GFPCre:IRES:HSVTK mRNA encodes functional GFPCre and HSVTK proteins.

2.2 Evaluation of mutagenesis step: In the application of the Cre-trap Cloning method, the GFPCre:IRES:HSVTK cassette is targeted to both alleles of the gene of interest (Example 3). This is important as mutants are selected for loss of gene expression based on the loss of HSVTK activity (resistance to ganciclovir). If the GFPCre:IRES:HSVTK cassette were present as a single copy, one would expect the majority of ganciclovir resistant clones to be due to mutations in the HSVTK gene. To test the mutagenesis step, the BW-5 cell line, which has a single copy of the GFPCre:IRES:HSVTK cassette, is reinfected to generate clones with two independently integrated copies of this cassette. These clones are subjected to chemical mutagenesis with methanesulfonic acid ethyl ester (EMS) followed by isolation of ganciclovir resistant clones. Resistance to ganciclovir results from mutations of both copies of the HSVTK gene, both LTRs or both copies of a gene encoding a trans-acting factor required to promote expression from the LTR.

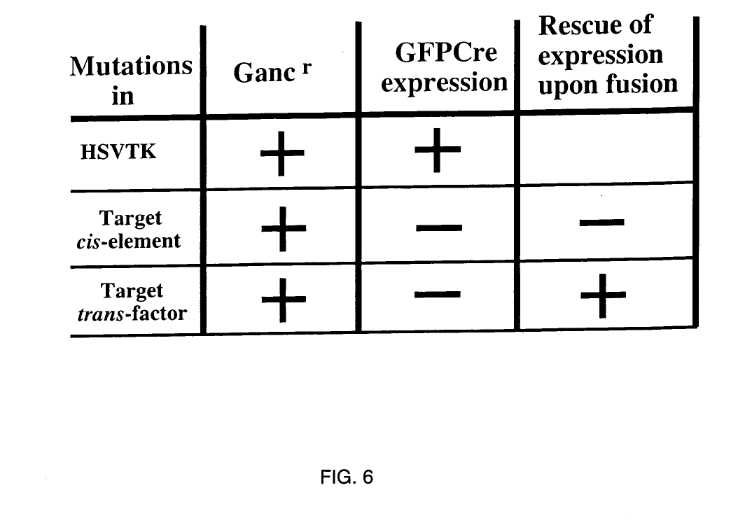

Mutant ganciclovir-resistant clones that exhibit GFPCre expression, as assayed by flow cytometry, would be expected to have mutations in the HSVTK gene and are excluded from further analyses. The Gal4 cDNA are transiently expressed in those ganciclovir resistant clones that have lost GFPCre expression. Mutant cell lines (hereafter referred to as BWm cells) in which Gal4 binding to the UAS promotes expression of functional GFPCre and HSVTK proteins are used for the analyses described in steps 2.3 and 2.4. It should be noted that in the application of this method to a target gene of interest, cells that have mutations in both HSVTK genes and those with mutations in target gene cis-acting elements (see Example 3, FIG. 6) are eliminated. However, for the purposes of testing the method as outlined in steps 2.3 and 2.4, cells with mutations in both LTRs or both alleles of genes encoding trans-acting factors that promote expression from the LTRs can be used.

2.3 Critical parameters for Cre Trap clone capture: To establish the parameters required to capture a clone of interest during an initial screen of the method, the BWm cells generated by mutagenesis in step 2.2 are used. The Gal4 cDNA is subcloned into pCT.1 generating pCT.1-Gal4 (pCT.1 AMP-Gal4). pCT.1^{AMP}-Gal4 is transiently transfected into the BWm cells followed by recovery at different times post-transfection to determine the minimal time for Gal4 induced expression of GFPCre and maximal conversion of pCT.1^{AMP}-Gal4 (equimolar pCT.1^{AMP}-Gal4 and pCT.1^{CAT} Gal4).

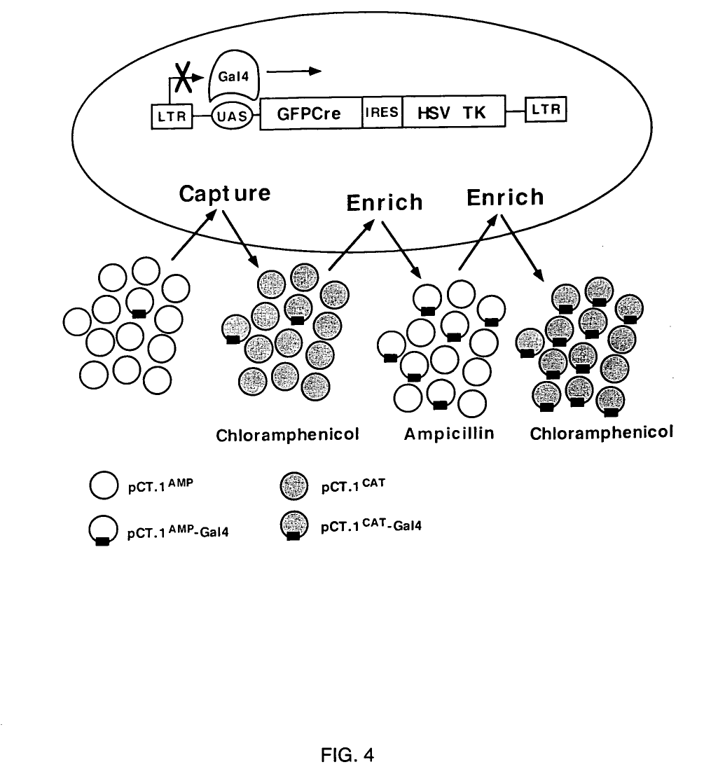

To assess the capacity of the Cre Trap Cloning method to capture a clone of interest, pCT.1^{AMP}-Gal4 is progressively diluted into pCT.1^{AMP} keeping the total amount of plasmid constant (FIG. 4). Plasmid mixtures are transiently transfected into BWm cell lines followed by recovery of plasmids at the optimal time defined above and analysis for the pCT.1^{CAT}-Gal4 conversion product. The capture step can be optimized such that pCT.1^{CAT}-Gal4 can be isolated from an initial pCT.1^{AMP}-Gal4/pCT.1^{AMP} mixture at 10⁻⁴ and preferably at 10⁻⁵ to 10⁻⁶. The capacity of pCT.1^{AMP}-Gal4 and pCT.1^{CAT}-Gal4 to replicate episomally can improve the capture efficiency of rare clones. Due to the reversibility of the Cre mediated inversion, there is a 50 percent chance of recovering a single copy of a pCT.1-Gal4 as pCT.1^{CAT}-Gal4 from a cell expressing GFPCre. However, at five copies the probability of one being in the pCT.1^{CAT}-Gal4 configuration is greater than 95%.

Critical parameters for Cre Trap clone enrichment: The initial transfection yields a heterogeneous set of clones because a single cell takes up a heterogeneous set of plasmids (FIG. 4). To enrich this pool for clones of interest, advantage is taken of the fact that modification of pCT.1^{AMP} to pCT.1^{CAT} is reversible due to the regeneration of functional loxP sites (FIG. 1). The general enrichment step is to retransfect the BWm cell with the pCT.1^{CAT} plasmid clones from the capture transfection followed by plasmid recovery and selection for plasmids now in the pCT.1^{AMP} configuration (FIG. 4). The enrichment process is tested using the lowest ratio of pCT.1^{AMP}-Gal4/pCT.1^{AMP} mixture, which permits clone capture. Initially recovered plasmids are enriched through sequential rounds of transfection into the BWm cell lines alternating selection for plasmids in the pCT.1^{CAT} or pCT.1^{AMP} configurations (FIG. 4). The efficiency of the enrichment process is assayed by determining the pCT.1-Gal4/pCT.1 ratio after each step. When the identity of a clone of interest is unknown, enrichment is assayed by simplification of a fingerprint digest (digestion with mixtures of frequent cutting restriction enzymes).

### EXAMPLE 3

### Application of Cre Trap Cloning to a Target Gene

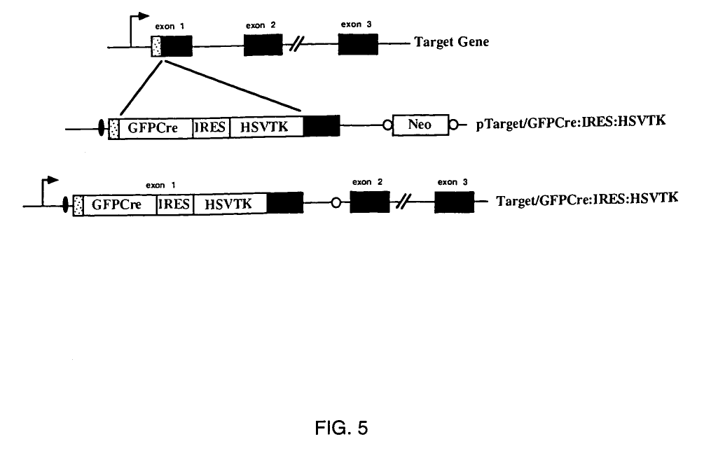

3.1 Generation of cell lines with GFPCre:IRES:HSVTK targeted loci: Standard gene targeting techniques are used to target the GFPCre:IRES:HSVTK cassette to both alleles of the target gene of interest in cell lines that express this gene (FIG. 5). The gene targeting places the GFPCre:IRES:HSVTK cassette in the locus such that the ATG normally used as the translation start for the target gene of interest is used as the translational start for GFPCre (FIG. 5). The gene targeting inserts the GFPCre:IRES:HSVTK cassette into the appropriate exon without deleting any portion of the gene, thus minimizing the possibility of disrupting cis-acting regulatory elements. The Neomycin gene flanked by FRT sites is deleted from the targeted allele by transient expression of the mutant Flp recombinase protein (Flpe) which has enhanced function at 37°. FRT is a DNA sequence recognized by the Flpe protein much in the same way that the loxP sites are recognized by Cre. The GFPCre:IRES:HSVTK cassette is targeted to the second allele in a similar manner.

3.2 Isolation of genes encoding proteins that regulate target gene expression: Cells expressing the GFPCre:IRES:HSVTK cassette from the target gene of interest are chemically mutagenized with EMS and selected in ganciclovir as described above. Ganciclovir resistant clones result from mutations in both HSVTK genes, mutations of target gene cis-acting elements on both alleles or mutations of both alleles encoding a trans-acting factor that regulates expression of the gene of interest. Cells with mutations in trans-acting factors are distinguished from those with mutations in the HSVTK gene or cis-acting elements by flow cytometric analyses and rescue of GFPCre:IRES:HSVTK cassette expression upon fusion to the parent cell line (FIG. 6). Clones determined to have mutations in trans-acting factors are fused to each other to identify genetic complementation groups having mutations in distinct trans-acting factor genes.

cDNA libraries are generated in the pCT.1 vector from the parent cell lines. cDNA clones encoding trans-acting factors that rescue target gene expression are isolated through transient transfection of the pCT.1 libraries into the different complementation group mutants. The initial clone capture and enrichment is carried out using the parameters defined in Example 2. As a cloning efficiency control, limiting amounts of pCT.1-Gal4 are added to each library as the UAS is introduced just upstream of the first exon allowing for the isolation of pCT.1-Gal4 (FIG. 5). In addition to the recovery of pCT.1-Gal4, enrichment is assayed during progressive rounds of transfection by monitoring the complexity of fingerprint digests, which simplifies with decreased clone heterogeneity. Once the complexity of the fingerprint digests reaches a steady state, individual clones are isolated and tested for their ability to rescue target expression in mutant cell lines from the different genetic complementation groups.

Various other examples will be apparent to the person skilled in the art after reading the present disclosure without departing from the spirit and scope of the invention. It is intended that all such other examples be included within the scope of this disclosure and the appended claims.

### SCIENTIFIC REFERENCES

1. Wang, M. M. and Reed, R. R., Molecular Cloning of the Olfactory Neuronal Transcription Factor Olf-1 by Genetic Selection in Yeast. Nature, 1993. 364: 121-6.

2. Luo, Y., Vijaychander, S., Stile, J. & Zhu, L. Cloning and Analysis of DNA-Binding Proteins by Yeast One-hybrid and One-two-hybrid Systems, Biotechniques, 1996.20: 564-8

3. Hasegawa, T., Takeuchi, A., Miyaishi, O., Isobe, K. and de Crombrugghe, B. Cloning and Characterization of a Transcription Factor that Binds to the Proximal Promoters of the Two Mouse Type I Collagens. J. Biol. Chem., 1997. 272: 4915-4923

4. Sauer, B. and Henderson, N., Cre-stimulated Recombination at loxP-containing DNA Sequences Placed into the Mammalian Genome. Nucleic Acids Res., 1989.17: 147-61

5. Sauer, B., Functional Expression of the cre-lox site-specific Recombination System in the Yeast Saccharomyces Cerevisiae. Mol. Cell Biol., 1987. 7: 2087-2096

6. Sauer, B. and Henderson, N., Site-Specific DNA Recombination in Mammalian Cells by the Cre recombinase of Bacteriophage P1. Proc. Natl. Acad. Sci. USA, 1988. 85: 5166-70

7. Matthew, M. and Hedges, R. W., Analytical Isoelectric Focusing of R Factor-Determined Beta-lactamases; Correlation with Plasmid Compatibility., J. Bacteriol., 1976 125: 713-8

8. Hesse, J. E., Lieber, M. R., Gellert, M., Mizuuchi, K., Extrachromosomal DNA Substrates in Pre-B Cells Undergo Inversion or Deletion at Immunoglobulin V-(D)-J joining Signals. Cell, 1987 49: 775-83

9. Russell, D. R. and Bennett, G. N., Characterization of the Beta-lactamase Promoter of pBR322., Nucleic Acids Res., 1981 9: 2517-33

10. Kilby, N. J., Snaith, M. R. and Murray, J. A., Site-specific Recombinases: Tools for Genome Engineering. Trends Genet. 1993. 9: 413-21

11. Gassmann, M., Donoho, G. and Berg, P., Maintenance of an Extrachromosomal Plasmid Vector in Mouse Embryonic Stem Cells. Proc. Natl. Acad. Sci. USA, 1995. 92: 1292-6

12. Seed, B. and Aruffo, A., Molecular Cloning of the CD2 antigen, the T-cell Erythrocyte Receptor, by a Rapid Immunoselection Procedure. Proc. Natl. Acad. Sci. USA, 1987.84: 3365-9

13. Scott, J. R., Regulation of Plasmid Replication. Microbiol. Rev. 1984. 48: 1-23

14. Hill, G. J. 2^{nd}, Shine, T. E., Hill, H. Z. and Miller, C., Failure of Amygdalin to Arrest B16 Melanoma and BW5147 AKR Leukemia. Cancer Res. 1976, 36: 2102-7

15. Luciw, P. A., Bishop, J. M., Varmus, H. E. and Capecchi, M. R., Location and Function of Retroviral and SV40 Sequences That Enhance Biochemical Transformation After Microinjection of DNA. Cell, 1983. 33: 705-16

16. Gagneten, S., Le, Y., Miller, J. and Sauer, B., Brief Expression of a GFP cre Fusion Gene in Embryonic Stem Cells Allows Rapid Retrieval of Site-Specific Genomic Deletions. Nucleic Acids Res. 1997. 25: 3326-31

17. Nagy, A., Cre Recombinase: The Universal Reagent for Genome Tailoring, Genesis, 2000, 26: 99-109

18. Wigler, M., Silverstein, S., Lee, L.-S., Pellicer, A., Cheng, Y.-C., and Axel, R. Transfer of Purified Herpes Virus Thymidine Kinase Gene to Cultured Mouse Cells. Cell, 1977. 11: 223-232

What is claimed is:

1. Expression vector pCT.1 as shown in FIG. 1.

2. A method for isolating genes encoding proteins that regulate the expression of target genes of interest comprising the steps of:
a. modifying a target gene of interest such that it encodes the Cre recombinase and Herpes simplex virus (HSV) thymidine kinase (TK) proteins,
b. subjecting the modified target gene to mutagenesis and selecting for cells which have lost target gene expression by virtue of their resistance to ganciclovir which kills cells expressing HSV TK,
c. transiently transfecting with a cDNA library from the parent cell line in expression vector pCT.1 said cells that have lost target gene expression due to mutations in genes encoding trans-acting factors,
d. expressing in said transfected cells a cDNA that complements the genetic mutation that results in expression of the target gene and production of Cre recombinase which modifies pCT.1, and
e. isolating said modified pCT.1 from pCT.1 plasmids that do not activate target gene expression.
